(19) United States
(12) Patent Application Publication
(10) Pub. No.: US 2008/0312340 A1
(43) Pub. Date: Dec. 18, 2008

(54) Process for Preparing Dispersions

(75) Inventors: David Hobson, Belper (GB); Alex Psaila, Caterham (GB)

Correspondence Address: THE LUBRIZOL CORPORATION;ATTN: DOCKET CLERK, PATENT DEPT., 29400 LAKELAND BLVD., WICKLIFFE, OH, 44092, US

(73) Assignee: The Lubrizol Corporation, Wickliffe, OH (US)

(21) Appl. No.: 12/092,109

(22) PCT Filed: Nov. 9, 2006
(86) PCT No.: PCT/US06/60707

§ 371 Date: Jun. 26, 2008

Related U.S. Application Data
(60) Provisional application No. 60/735,961, filed on Nov. 10, 2005.

(51) Int. Cl. A61K 47/02; C10M 125/10; A01N 25/02; A61P 43/00

(52) U.S. Cl. 514/769; 508/154; 508/180; 508/165; 508/171

(57) ABSTRACT

The invention provides a process for preparing a dispersion of particles, by reducing in particle size at least one component by agitating the component in the presence of (i) a plurality of beads; (ii) a powder; (iii) a surfactant; and (iv) a liquid medium to form a dispersion, wherein the particles are uniformly dispersed in the liquid medium. The invention further provides for the use of the dispersion of particles.

### FIELD OF INVENTION

The present invention relates to a process for preparing a dispersion of particles by reducing in particle size at least one component by agitating the component in the presence of (i) a plurality of beads; (ii) a powder; (iii) a surfactant; and (iv) a liquid medium to form a dispersion, wherein the particles are uniformly dispersed in the liquid medium. The invention further provides for the use of the dispersion of particles.

### BACKGROUND OF THE INVENTION

In recent years attempts have been made to produce dispersions of particles. Typically dispersions of particles are unstable and may agglomerate quickly if the dispersion system is not sufficiently balanced for HLB, phase interface control, solids content and the like. Further dispersions are commonly prepared by employing chemical processes. However, chemical processes require complex production facilities and are expensive.

Attempts have been made to produce dispersions of particles with grinding technology, such as wet grinding, bead mills etc. The grinding technology employed in the art, results in dispersions with mean particle sizes or less than 300 nm being difficult to achieve. However, Schaer has reported in “Coating, 38 (1), 18-2), 2005,” a monodisperse dispersion by modifying the particle size with functional molecules, such as, carboxylic acids, β-diketones, or silanes. The functional molecules enable crosslinking in a matrix or to facilitate the formation of the dispersion.

However, none of the dispersions known in the art provide a process for preparing a dispersion with reduced operating conditions and capable of lowering the mean particle size of the dispersion particles, or a reduced agitation time, or combinations thereof. The present invention provides such a process for preparing such a dispersion. The invention further provides for the use of the dispersion in a wide variety of applications.

### SUMMARY OF THE INVENTION

The present invention in one embodiment provides a process for preparing a dispersion of particles, comprising: reducing in particle size at least one component by agitating the component in the presence of (i) a plurality of beads; (ii) a powder; (iii) a surfactant; and (iv) a liquid medium to form a dispersion, wherein the particles are uniformly dispersed in the liquid medium.

In one embodiment the invention provides a composition comprising: (a) a dispersion of particles obtained/obtainable from the process disclosed herein; and (b) at least one member of the group consisting of an oil of lubricating viscosity, a grease thickener a liquid fuel, other performance additives, and mixtures thereof.

In one embodiment the invention provides a lubricating composition comprising an oil of lubricating viscosity, and a dispersion of particles disclosed herein.

In one embodiment the invention provides a lubricating composition comprising an oil of lubricating viscosity, a grease thickener, and a dispersion of particles disclosed herein.

In one embodiment the invention provides a fuel composition comprising a liquid fuel, and a dispersion of particles disclosed herein.

### DETAILED DESCRIPTION OF THE INVENTION

The present invention provides a process for preparing a dispersion of particles as disclosed above.

As used herein the term “powder” is used in the ordinary meaning i.e. a powder is a solid substance in the form of loosely bound or loosely associated particles.

As used herein the term “bead” is used in the ordinary meaning i.e. a bead is a solid substance in which particles have been fused (for example, melted or strongly bound) together.

In one embodiment the agitating process is other than a native grinding process. As used herein the term “native” means that the bead and component being agitated have the same formulae and crystal structures e.g. employ dolomite as the component and the bead.

The dispersion of particles may have an average mean particle size ranging from at least about 10 nm to less than about 1 μm, or from about 20 nm to about 750 μm, or from about 30 nm to about 300 μm, or from about 35 nm to about 220 μm.

In one embodiment the invention is other than a water-containing emulsion.

As used herein the term “free of” for all chemistry disclosed herein except for the metal base, as used in the specification and claims, defines the absence of a material except for the amount which is present as impurities, e.g., a trace amount or a non-effective amount. Typically in this embodiment, the amount present will be less than about 0.05% or less than about 0.005 wt % by weight of the dispersion.

As a person skilled in the art will appreciate, impurities in a metal base are typically about 1 wt % to about 3 wt % of the metal base. The reason for the impurities being typically about 1 wt % to about 3 wt % of the metal base is believed to be due to mining processes. Typically the major impurities in the metal base include calcium carbonates, silica or silicates.

In different embodiments the dispersion may be opaque or semi-translucent or translucent or transparent, or any gradation between such descriptions.

### Agitating Process

Agitating the component may be carried out by a number of techniques including wet or dry processes for particle size reduction of the component. Examples of suitable agitating process further comprise ultra-sonic wave treatment, milling, grinding, crushing or mixtures thereof. In one embodiment agitating may be carried out by grinding or milling.

Agitating processes carried out by grinding or milling may employ a rotor stator mixer, a vertical bead mill, a horizontal bead mill, basket milling, ball mill, pearl milling or mixtures thereof. In one embodiment, the agitating processes comprise using a vertical or horizontal bead mill.

In different embodiments the agitating processes may be carried out in a vertical or horizontal bead mill. Either bead mill processes cause the reduction of particle size of the metal base by high energy collisions of the metal base with at least one bead; and/or other metal base agglomerates, aggregates, solid particles; or mixtures thereof.

The vertical or horizontal bead mill typically contains beads present at least about 40 vol %, or at least about 60 vol % of the mill. A range include for example about 60 vol % to about 95 vol %.

The agitating conditions for preparing the dispersion of the invention, may include agitating for a period of time ranging from about 30 seconds to about 48 hours, or from about 2 minutes to about 24 hours, or from about 5 minutes to about 16 hours, or from about 10 minutes to about 5 hours; and at pressures that may range from about 0 kPa to about 500 kPa (about 0 mm Hg to about 3750 mm Hg), or from about 0 kPa to about 266 kPa (about 0 mm Hg to about 2000 mm Hg), or from about 10 kPa to about 200 kPa (about 75 mm Hg to about 1500 mm Hg), or from 1 about 10 kPa to about 133 kPa (about 75 mm Hg to about 1000 mm Hg); and at a temperature that may range from about 0° C. to about 100° C., or from about 10° C. to about 85° C.

### Beads

The beads typically have a mean particle size and mass greater than the desired mean particle size of the component. In some instances the beads are a mixture or different mean particle size. The beads used in the grinding may be of materials known to those skilled in the art, such as metal, ceramic, glass, stone, or composite materials.

The mean particle size of the beads may range from about 10 μm to about 10 mm, or from about 20 μm to about 5 mm, or from about 0.05 mm to about 4 mm, or from about 0.8 mm to about 4 mm.

In one embodiment the process further comprises removing the beads from the dispersion of particles. Removing the beads from the dispersion of particles may be carried out by centrifuging, sieving, or other known extraction or purification techniques.

### Powder and at Least One Component

The powder and component may be the same, similar or different chemical species. In one embodiment the powder and component are chemically distinct from each other.

In one embodiment the powder and the component are both inorganic. In one embodiment the powder is inorganic and the component is organic. In one embodiment the powder and/or component are metal bases, or mixtures thereof.

In one embodiment the dispersion comprises a mixture of one powder and one component.

In one embodiment the dispersion comprises a mixture of at least two powders and at least one component.

The metal base generally comprises at least one of oxides, hydroxides or carbonates. Examples of a suitable metal base include magnesium hydroxide, calcium hydroxide, calcium carbonate, magnesium carbonate, calcium oxide, magnesium oxide, cerium oxide, iron oxide, sodium carbonate, sodium bicarbonate, potassium carbonate, potassium bicarbonate, potassium hydroxide, sodium hydroxide, anhydrous lithium hydroxide, lithium hydroxide monohydrate, lithium carbonate, lithium oxide, copper acetate, or mixtures thereof. In one embodiment of the invention the metal base is present in a mixture, for instance, dolmitic lime, which is commercially available.

In one embodiment the component comprises at least one of lubricant or fuel technology additives, pharmaceutical active agents, agrochemical active agents, personal care active agents, or mixtures thereof. In one embodiment the component comprises an organic compound, (such as, a nitrogen containing base), pharmaceutical active agents, agrochemical active agents, personal care active agents, or mixtures thereof. Examples of suitable an organic compound include aminoguanidine, aminoguanidine carbonate, aminoguanidine bicarbonate, or mixtures thereof.

The choice of species for the powder and the component may be determined by the specific nature of the end use of the dispersion of particles.

In one embodiment the powder has a mean particle size less than that of the component when agitating processes are initiated.

In one embodiment the powder is harder (as defined by the Mohs Scale (ranges from 1-10), Modified Mohs Scale (ranges from 1 to 15), or Knoop Scale (assumes load of 100 g)) than the component.

In one embodiment the dispersion of particles formed by the invention, contains both the powder and the component. Typically the powder and component are compatible with the desired use of the dispersion of particles.

In one embodiment the dispersion of particles produced at the end of the agitating process may be substantially free of, to free of the powder. Removing the powder from the dispersion of particles may be carried out by centrifuging, sieving, or other known extraction or purification techniques.

### Powder

The powder is believed to work in combination with the beads to reduce the particle size of the component by agitating, that is, by any one or more of various physical processes, i.e., physical processing steps.

Before agitating, the powder may have a mean particle size ranging from about 0.01 nm to about 20 mm, or from about 1 nm to about 1 μm, or from about 10 nm to about 50 μm.

During agitating, the powder loose particles typically de-agglomerate. During the agitating process, the powder may have a mean particle size ranging from about 20 nm to about 45 μm, or from about 40 nm to about 40 μm.

### Surfactant

The surfactant includes an ionic (cationic or anionic) or non-ionic compound. Generally, the surfactant stabilises the dispersion of the metal base in the organic medium.

Suitable surfactant compounds include those with a hydrophilic lipophilic balance (HLB) ranging from about 1 to about 40, or about 1 to about 20, or about 1 to about 18, or about 2 to about 16, or about 2.5 to about 15. In different embodiments the HLB may be about 11 to about 14, or less than about about 10 such as about 1 to about 8, or about 2.5 to about 6. Combinations of surfactants may be used with individual HLB values outside of these ranges, provided that the composition of a final surfactant blend is within these ranges. When the surfactant has an available acidic group, the surfactant may become the metal salt of the acidic group and where the metal is derived from the metal base.

Examples of surfactants suitable for the invention are disclosed in McCutcheon's Emulsifiers and Detergents, 1993, North American & International Edition. Generic examples include alkanolamides, alkylarylsulphonates, amine oxides, poly(oxyalkylene) compounds, including block copolymers comprising alkylene oxide repeat units (e.g., Pluronic™), carboxylated alcohol ethoxylates, ethoxylated alcohols, ethoxylated alkyl phenols, ethoxylated amines and amides, ethoxylated fatty acids, ethoxylated fatty esters and oils, fatty esters, glycerol esters, glycol esters, imidazoline derivatives, phenates, lecithin and derivatives, lignin and derivatives, monoglycerides and derivatives, olefin sulphonates, phosphate esters and derivatives, propoxylated and ethoxylated fatty acids or alcohols or alkyl phenols, sorbitan derivatives, sucrose esters and derivatives, sulphates or alcohols or ethoxylated alcohols or fatty esters, polyisobutylene succinicimide and derivatives.

In one embodiment the surfactant comprises polyesters as defined in column 2, line 44 to column 3, line 39 of U.S. Pat. No. 3,778,287. Examples of suitable polyester surfactants are prepared in U.S. Pat. No. 3,778,287 as disclosed in Polyester Examples A to F (including salts thereof).

In one embodiment the surfactant is a hydrocarbyl substituted aryl sulphonic acid (or sulphonate) of an alkali metal, alkaline earth metal or mixtures thereof. The aryl group of the aryl sulphonic acid may be phenyl or naphthyl. In one embodiment the hydrocarbyl substituted aryl sulphonic acid comprises alkyl substituted benzene sulphonic acid.

The hydrocarbyl (especially an alkyl) group typically contains about 8 to about 30, or about 10 to about 26, or about 10 to about 15 carbon atoms. In one embodiment the surfactant is a mixture of C₁₀ to C₁₅ alkylbenzene sulphonic acids. Examples of sulphonates include dodecyl and tridecyl benzene sulfonates or condensed naphthalenes or petroleum sulfonates, as well as sulphosuccinates and derivatives.

In one embodiment the surfactant is in the form of a neutral or overbased surfactant, typically salted with an alkali or alkaline earth metal. The alkali metal includes lithium, potassium or sodium; and the alkaline earth metal includes calcium or magnesium. In one embodiment the alkali metal is sodium. In one embodiment the alkaline earth metal is calcium.

In one embodiment the surfactant is a derivative of a polyolefin. Typical examples of a polyolefin include polyisobutene; polypropylene; polyethylene; a copolymer derived from isobutene and butadiene; a copolymer derived from isobutene and isoprene; or mixtures thereof.

Typically the derivative of a polyolefin comprises a polyolefin-substituted acylating agent optionally further reacted to form an ester and/or aminoester. The acylating agent may be prepared from carboxylic reactants (which when reacted with a polyolefin give the desired acylating agent, i.e. substrate for the surfactant). The carboxylic reactants include functional groups, such as a carboxylic acid or anhydride thereof. Examples of carboxylic reactants include an alpha, beta-unsaturated mono- or polycarboxylic acid, anhydride ester or derivative thereof. Examples of carboxylic reactants thus include (meth) acrylic acid, methyl (meth)acrylate, maleic acid or anhydride, fumaric acid, itaconic acid or anhydride, or mixtures thereof, each of which may typically be in the form of the saturated materials (e.g. succinic anhydride) after reaction with the polyolefin.

In one embodiment the polyolefin is a derivative of polyisobutene with a number average molecular weight of at least 250, 300, 500, 600, 700, or 800, to 5000 or more, often up to 3000, 2500, 1600, 1300, or 1200. Typically, less than about 5% by weight of the polyisobutylene used to make the derivative molecules have M̅n̅ less than about 250, more often the polyisobutylene used to make the derivative has M̅n̅ of at least about 800. The polyisobutylene used to make the derivative preferably contains at least about 30% terminal vinylidene groups, more often at least about 60% or at least about 75% or about 85% terminal vinylidene groups. The polyisobutylene used to make the derivative may have a polydispersity, M̅w̅/ M̅n̅ greater than about 5, more often from about 6 to about 20.

In various embodiments, the polyisobutene is substituted with succinic anhydride, the polyisobutene substituent having a number average molecular weight ranging from about 1,500 to about 3,000, or about 1,800 to about 2,300, or about 700 to 1 about 700, or about 800 to about 1000. The ratio of succinic groups per equivalent weight of the polyisobutene typically ranges from about 1.3 to about 2.5, or about 1.7 to about 2.1, or about 1.0 to about 1.3, or about 1.0 to about 1.2.

In one embodiment the surfactant is polyisobutenyl-dihydro-2,5-furandione ester with pentaerythritol or mixtures thereof. In one embodiment the surfactant is a polyisobutylene succinic anhydride derivative such as a polyisobutylene succinimide or derivatives thereof. In one embodiment the surfactant is substantially free to free of a basic nitrogen.

Other typical derivatives of polyisobutylene succinic anhydrides include hydrolysed succinic anhydrides, esters or diacids. Polyisobutylene succan derivatives are preferred to make the metal base dispersions. A large group of polyisobutylene succinic anhydride derivatives are taught in U.S. Pat. No. 4,708,753, and U.S. Pat. No. 4,234,435.

In another embodiment the surfactant comprises a salixarene (or salixarate if in the form of a metal salt). The salixarene is defined as an organic substrate of a salixarate. The salixarene may be represented by a substantially linear compound comprising at least one unit of the formulae (I) or (II):

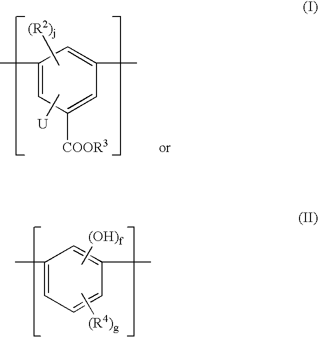

each end of the compound having a terminal group of formulae (III) or (IV):

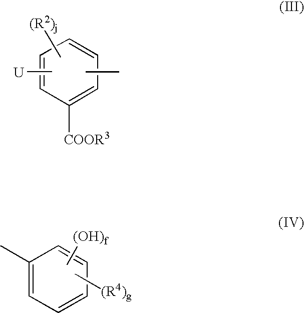

such groups being linked by divalent bridging groups, which may be the same or different for each linkage; wherein f is about 1, 2 or 3, in one embodiment about 1 or 2; R² is hydroxyl or a hydrocarbyl group and j is about 0, 1, or 2; R³ is hydrogen or a hydrocarbyl group; R⁴ is a hydrocarbyl group or a substituted hydrocarbyl group; g is about 1, 2 or 3, provided at least one R⁴ group contains 8 or more carbon atoms; and wherein the compound on average contains at least one of unit (I) or (III) and at least one of unit (II) or (IV) and the ratio of the total number of units (I) and (III) to the total number of units of (II) and (IV) in the composition is about 0.1:1 to about 2:1.

The U group in formula (I) and (III) may be an —OH or an —NH₂ or —NHR¹ or N(R¹)₂ group located in one or more positions ortho, meta, or para to the —COOR³ group. R¹ is a hydrocarbyl group containing 1 to 5 carbon atoms. When the U group comprises a —OH group, formulae (I) and (III) are derived from 2-hydroxybenzoic acid (often called salicylic acid), 3-hydroxybenzoic acid, 4-hydroxybenzoic acid or mixtures thereof. When U is a —NH)₂ group, formulae (I) and (III) are derived from 2-aminobenzoic acid (often called anthranilic acid), 3-aminobenzoic acid, 4-aminobenzoic acid or mixtures thereof.

The divalent bridging group, which may be the same or different in each occurrence, includes an alkylene or methylene bridge such as —CH₂— or —CH(R)— and an ether bridge such as —CH₂OCH₂— or —CH(R)OCH(R)— where R is an alkyl group having 1 to 5 carbon atoms and where the methylene and ether bridges are derived from formaldehyde or an aldehyde having 2 to 6 carbon atoms.

Often the terminal group of formulae (III) or (IV) further contains 1 or 2 hydroxymethyl groups ortho to a hydroxy group. In one embodiment of the invention hydroxymethyl groups are present. In one embodiment of the invention hydroxymethyl groups are not present. A more detailed description of salixarene and salixarate chemistry is disclosed in EP 1 419 226 B1, including methods of preparation as defined in Examples 1 to 23 (page 11, line 42 to page 13, line 47).

In one embodiment the surfactant is substantially free of, to free of, a fatty acid or derivatives thereof, such as esters. In one embodiment the surfactant is other than a fatty acid or derivatives thereof.

In one embodiment the surfactant comprises at least of hydrocarbyl substituted aryl sulphonic acids, derivatives of polyolefins, polyesters or salixarenes (or salixarates).

In different embodiments the surfactant is substantially free of to free of, phospholipids, (such as lecithin) and/or amino acids (such as sarcosines).

In one embodiment the surfactant has a molecular weight of less than 1000, in another embodiment less than about 950, for example, about 250, about 300, about 500, about 600, about 700, or about 800.

The amount of surfactant and the total amount of powder and the component in the dispersion may vary as is shown in Table 1, the balance being the organic medium and optionally water. In one embodiment the amount of the organic medium present in the dispersion varies from about 25 wt % to about 55 wt %.

TABLE 1

| | Embodiments (wt % of dispersion) | | | |
|---|---|---|---|---|
| Additive | 1 | 2 | 3 | 4 |
| Σ (wt % of Powder and wt % of | 17-90 | 25-80 | 35-70 | 40-65 |
| Component) | | | | |
| Surfactant | 0.01-30 | 1-30 | 2-30 | 5-25 |

### Demulsifiers

In one embodiment the dispersion further comprises demulsifiers, or mixtures thereof. Examples of demulsifiers include trialkyl phosphates, polyethylene glycols, polyethylene oxides, polypropylene oxides and (ethylene oxide-propylene oxide) polymers, alkoxylated alkyl phenol resins or mixtures thereof.

In one embodiment the dispersion further comprises a co-ordination compound, such as, ferrocene (cyclopentadienyl based), carboxylates or sulphonates.

### Organic Medium

The organic medium may comprise an oil of lubricating viscosity, a liquid fuel, a hydrocarbon solvent, pharmaceutical or agrochemical carrier fluids (such as, digestible oil, or fatty acid, or esters thereof) or mixtures thereof. Typically the organic solvent comprises an oil of lubricating viscosity or a liquid fuel.

Optionally the organic medium contains water, typically up to about wt %, or about 2 wt % or about 3 wt % of the dispersion. In different embodiments the organic medium is substantially free of, to free of, water.

### Oils of Lubricating Viscosity

In one embodiment the organic medium comprises an oil of lubricating viscosity. Such oils include natural and synthetic oils, oil derived from hydrocracking, hydrogenation, and hydrofinishing, unrefined, refined and re-refined oils and mixtures thereof.

Unrefined oils are those obtained directly from a natural or synthetic source generally without (or with little) further purification treatment.

Refined oils are similar to the unrefined oils except they have been further treated in one or more purification steps to improve one or more properties. Purification techniques are known in the art and include solvent extraction, secondary distillation, acid or base extraction, filtration, percolation and the like.

Re-refined oils are also known as reclaimed or reprocessed oils, and are obtained by processes similar to those used to obtain refined oils and often are additionally processed by techniques directed to removal of spent additives and oil breakdown products.

Natural oils useful in making the inventive lubricants include animal oils, vegetable oils (e.g., castor oil, lard oil), mineral lubricating oils such as liquid petroleum oils and solvent-treated or acid-treated mineral lubricating oils of the paraffinic, napthenic or mixed paraffinic-naphthenic types and oils derived from coal or shale or mixtures thereof.

Synthetic lubricating oils are useful and include hydrocarbon oils, such as, polymeric tetrahydrofurans, polymerised and interpolymerised olefins (e.g., polybutylenes, polypropylenes, propyleneisobutylene copolymers), poly(1-hexenes), poly(1-octenes), poly(1-decenes), and mixtures thereof, alkyl-benzenes (e.g. dodecylbenzenes, tetradecylbenzenes, dinonylbenzenes, di-(2-ethylhexyl)-benzenes); polyphenyls (e.g., biphenyls, terphenyls, alkylated polyphenyls); alkylated diphenyl ethers and alkylated diphenyl sulphides and the derivatives, analogs and homologs thereof or mixtures thereof.

Other synthetic lubricating oils include. Synthetic oils may be produced by Fischer-Tropsch reactions and typically may be hydroisomerised Fischer-Tropsch hydrocarbons or waxes.

Oils of lubricating viscosity may also be defined as specified in the American Petroleum Institute (API) Base Oil Interchangeability Guidelines. The five base oil groups are as follows: Group I (sulphur content >0.03 wt %, and/or <90 wt % saturates, viscosity index 80-120); Group II (sulphur content ≦0.03 wt %, and ≧90 wt % saturates, viscosity index 80-120); Group III (sulphur content ≦0.03 wt %, and ≧90 wt % saturates, viscosity index ≧−120); Group IV (all polyalphaolefins (PAOs)); and Group V (all others not included in Groups I, II, III, or IV). The oil of lubricating viscosity comprises an API Group I, Group II, Group III, Group IV, Group V oil and mixtures thereof. Often the oil of lubricating viscosity is an API Group I, Group II, Group III, Group IV oil and mixtures thereof. Alternatively the oil of lubricating viscosity is often an API Group I, Group II, Group III oil or mixtures thereof.

### Liquid Fuel

The liquid fuel is normally a liquid at ambient conditions. The liquid fuel includes a hydrocarbon fuel, a biofuel (such as, bio-diesel), a nonhydrocarbon fuel, or a mixture thereof. The hydrocarbon fuel may be a petroleum distillate such as a gasoline as defined by ASTM (American Society for Testing and Materials) specification D4814 or a diesel fuel as defined by ASTM specification D975. In an embodiment the liquid fuel is a gasoline, and in another embodiment the liquid fuel is a leaded gasoline, or a nonleaded gasoline. In another embodiment the liquid fuel is a diesel fuel. The hydrocarbon fuel includes a hydrocarbon prepared by a gas to liquid process for example hydrocarbons prepared by a process such as the Fischer-Tropsch process. The nonhydrocarbon fuel includes an oxygen containing composition (often referred to as an oxygenate), an alcohol, an ether, a ketone, an ester of a carboxylic acid, a nitroalkane, or a mixture thereof. The nonhydrocarbon fuel includes methanol, ethanol, methyl t-butyl ether, methyl ethyl ketone, transesterified oils and/or fits from plants and animals such as rapeseed methyl ester and soybean methyl ester, and nitromethane. Mixtures of hydrocarbon and nonhydrocarbon fuels include gasoline and methanol and/or ethanol, diesel fuel and ethanol, and diesel fuel and a transesterified plant oil such as rapeseed methyl ester. In one embodiment the liquid fuel is a nonhydrocarbon fuel or a mixture thereof.

### INDUSTRIAL APPLICATION

The dispersion prepared by the process disclosed herein is suitable for a wide variety of applications. Examples of suitable applications include lubricant and fuel technology, in pharmaceutical, personal care or agrochemical compositions, in coating or floor covering technology, or in construction technology (such as, building products or bitumen).

In one embodiment the dispersion prepared by the process disclosed herein is suitable for agrochemical compositions. Typically, the agrochemical compositions comprise the dispersion as an active ingredient in a suitable organic medium, such as, digestible oil, or fatty acid, or ester thereof. The active ingredient may be inorganic or organic.

In one embodiment the dispersion prepared by the process disclosed herein is suitable for pharmaceutical compositions. Typically, the pharmaceutical compositions comprise the dispersion as an active ingredient in a suitable organic medium, such as, digestible oil, or fatty acid, or ester thereof. The active ingredient comprises any pharmacological agent or drug, (including inorganic or organic). The active ingredient may be taken orally, intravenous administered, or inhaled. The dispersion of pharmaceutical compositions has at least one of improved property over a non-dispersed equivalent active ingredient. The improved properties include bioavailability, ingestion time, morphologies, activity, controlled release or mixtures thereof. The dispersion of pharmaceutical compositions may also allow for the use of an active ingredient that is poorly soluble in water (typically a solubility of less than about 10 mg/ml)

In one embodiment the dispersion prepared by the process disclosed herein is suitable for fuel and is disclosed in more detail in co-pending U.S. application filed on the same date as this Application by inventors Hobson, Psaila and Spivey (U.S. Application No. 60/735,941). Typically the dispersion in a fuel is useful for numerous open or closed flame combustion systems. Suitable combustion systems include power stations, internal combustion engines, industrial and marine compression engines and turbines (commonly combusting a distillate, residual or heavy fuel oils).

The dispersion prepared by the process disclosed herein may be added to a fuel in ranges from about 1 ppm to about 10,000 ppm, or from about 20 ppm to about 7500 ppm, or from about 100 ppm to about 5000 ppm, or from about 200 ppm to about 3000 ppm.

In one embodiment the dispersion prepared by the process disclosed herein is suitable for lubricant technology. Examples of a lubricant include those suitable for transmission fluids, gear oils, hydraulic fluids or internal combustion engines. In another embodiment lubricant technology comprises greases. In one embodiment the lubricant is suitable for internal combustion engines.

Examples of suitable grease include a lithium soap grease made with a monocarboxylic acid, a complex soap grease, a lithium complex soap grease, a calcium soap grease, a low noise soap grease are (sometimes characterised by the lack of residual metal base particles above about 2 micrometres in diameter); a short fibre high soap content grease or mixtures thereof. In one embodiment the grease includes a lithium soap grease, in another embodiment a complex soap grease, in another embodiment a lithium complex soap grease, in another embodiment a low noise soap grease and in yet another embodiment a short fibre high soap content grease.

The low noise grease is known and typically used in rolling element bearing applications such as pumps or compressors. The complex soap grease is known and includes smooth or show grain. Furthermore, the complex grease contains a polycarboxylic acid typically a dicarboxylic acid. The short fibre high soap content grease is known and is often used in specialist applications.

A grease may be prepared by heating the dispersion of the invention with a known grease thickener. Grease thickener chemistry includes carboxylic acids, inorganic powders including clay, organo-clays, bentonite, fumed silica, calcite, carbon black, pigments, copper phthalocyanine or mixtures thereof. In one embodiment the grease may be prepared by heating the dispersion of the invention with a carboxylic acid and optionally one of the other known thickeners.

Suitable carboxylic acids include those containing about 2 to about 30 carbon atoms. The carboxylic acid may be a monocarboxylic acid, a polycarboxylic acid or mixtures thereof, and optionally further substituted with groups selected from a hydroxyl group, an ester and mixtures thereof. In one embodiment the carboxylic acid comprises a hydroxy substituted or an unsubstituted alkanoic acid. Typically, the carboxylic acids will have about 2 to about 30, or from about 4 to about 30, or from about 12 to about 24, or from about 16 to about 20 carbon atoms. In one embodiment the carboxylic acid is a hydroxystearic acid or esters of these acids such as 9-hydroxy-, 10-hydroxy- or 12-hydroxy-stearic acid, and especially 12-hydroxy stearic acid.

The polycarboxylic acid, especially dicarboxylic acids is present in a complex grease and suitable examples include iso-octanedioic acid, octanedioic acid, nonanedioic acid (azelaic acid), decanedioic acid (sebacic acid), undecanedioic acid, dodecanedioic acid, tridecanedioic acid, tetradecanedioic acid, pentadecanoic acid or mixtures thereof. In one embodiment the polycarboxylic acid is nonanedioic acid (azelaic acid) or mixtures thereof. In one embodiment the polycarboxylic acid is decanedioic acid (sebacic acid) or mixtures thereof.

Internal combustion engines include for example diesel fuelled engines, gasoline fuelled engines, natural gas fuelled engines or a mixed gasoline/alcohol fuelled engines. Suitable internal combustion engines include a marine diesel engine, aviation piston engines, low-load diesel engines, automobile and truck engines. In one embodiment internal combustion engines include a 2-stroke or a 4-stroke marine diesel engine, especially a 2-stroke engine.

The dispersion prepared by the process disclosed herein may be added to a lubricant in ranges from about 0.01 wt % to about 50 wt %, or from about 0.1 wt % to about 25 wt %, or from about 0.5 wt % to about 10 wt %, or about 0.75 wt % to about 5 wt %.

In one embodiment a lubricant or fuel composition containing the dispersion prepared by the process disclosed herein further comprises other performance additives. The other performance additives comprise at least one of metal deactivators, detergents, dispersants, friction modifiers, corrosion inhibitors, antioxidants, foam inhibitors, demulsifiers, pour point depressants, seal swelling agents, viscosity modifiers, dispersant viscosity modifiers, or mixtures thereof. Typically, a fully-formulated lubricant or fuel will contain one or more of these performance additives.

### Dispersants

Dispersants are often known as ashless-type dispersants because, prior to mixing in a lubricating oil composition, they do not contain ash-forming metals and they do not normally contribute any ash forming metals when added to a lubricant. Dispersants also include polymeric dispersants. Ashless type dispersants are characterised by a polar group attached to a relatively high molecular weight hydrocarbon chain. Typical ashless dispersants include N-substituted long chain alkenyl succinimides. Examples of N-substituted long chain alkenyl succinimides include polyisobutylene succinimide with number average molecular weight of the polyisobutylene substituent in the range 350 to 5000, or 500 to 3000. Succinimide dispersants and their preparation are disclosed, for instance in U.S. Pat. No. 4,234,435. Succinimide dispersants are typically the imide formed from a polyamine, typically a poly(ethyleneamine).

In one embodiment the invention further comprises at least one dispersant derived from polyisobutylene succinimide with number average molecular weight in the range 350 to 5000, or 500 to 3000. The polyisobutylene succinimide may be used alone or in combination with other dispersants.

In one embodiment the invention further comprises at least one dispersant derived from polyisobutylene, an amine and zinc oxide to form a polyisobutylene succinimide complex with zinc. The polyisobutylene succinimide complex with zinc may be used alone or in combination.

Another class of ashless dispersant is Mannich bases. Mannich dispersants are the reaction products of alkyl phenols with aldehydes (especially formaldehyde) and amines (especially polyalkylene polyamines). The alkyl group typically contains at least 30 carbon atoms.

The dispersants may also be post-treated by conventional methods by a reaction with any of a variety of agents. Among these are boron sources such as boric acid or borates, urea, thiourea, dimercaptothiadiazoles, carbon disulphide, aldehydes, ketones carboxylic acids, hydrocarbon-substituted succinic anhydrides, maleic anhydride, nitriles, epoxides, and phosphorus compounds.

### Detergents

The lubricant or fuel composition optionally further comprises neutral or overbased detergents. Suitable detergent substrates include sulphonates, salixarates, salicylates, carboxylates, phosphorus acid salts, mono- and/or di-thiophosphoric acid salts, phenates including alkyl phenates and sulphur coupled alkyl phenates, or saligenins.

In different embodiments, the lubricant or fuel composition further comprises at least one of sulphonates and phenates. When present, the detergents are typically overbased. The ratio of TBN delivered by the dispersion to that delivered by the detergent may range from 1:99 to 99:1, or 15:85 to 85:15.

### Antioxidant

Antioxidant compounds are known and include an amine antioxidant (such as an alkylated diphenylamine), a hindered phenol, a molybdenum dithiocarbamate, and mixtures thereof. Antioxidant compounds may be used alone or in combination.

The hindered phenol antioxidant often contains a secondary butyl and/or a tertiary butyl group as a sterically hindering group. The phenol group is often further substituted with a hydrocarbyl group and/or a bridging group linking to a second aromatic group. Examples of suitable hindered phenol antioxidants include 2,6-di-tert-butylphenol, 4-methyl-2,6-di-tert-butylphenol, 4-ethyl-2,6-di-tert-butylphenol, 4-propyl-2,6-di-tert-butylphenol or 4-butyl-2,6-di-tert-butylphenol 2,6-di-tert-butylphenol. In one embodiment the hindered phenol antioxidant is an ester and may include, e.g., Irganox™ L-135 from Ciba. A more detailed description of suitable ester-containing hindered phenol antioxidant chemistry is found in U.S. Pat. No. 6,559,105.

Suitable examples of molybdenum dithiocarbamates which may be used as an antioxidant include commercial materials sold under the trade names such as Vanlube 822™ and Molyvan™ A from R. T. Vanderbilt Co., Ltd., and Adeka Salkura-Lube™ S-100, S-165 and S-600 from Asahi Denka Kogyo K. K and mixtures thereof.

### Antiwear Agent

The lubricant or fuel composition optionally further comprises at least one antiwear agent. Examples of suitable antiwear agents include a sulphurised olefin, sulphur-containing ashless anti-wear additives, metal dihydrocarbyldithiophosphates (such as zinc dialkyldithiophosphates), thiocarbamate-containing compounds, such as thiocarbamate esters, thiocarbamate amides, thiocarbamic ethers, alkylene-coupled thiocarbamates, and bis(S-alkyldithiocarbamyl) disulphides.

The dithiocarbamate-containing compounds may be prepared by reacting a dithiocarbamic acid or salt with an unsaturated compound. The dithiocarbamate containing compounds may also be prepared by simultaneously reacting an amine, carbon disulphide and an unsaturated compound. Generally, the reaction occurs at a temperature from 25° C. to 125° C. U.S. Pat. Nos. 4,758,362 and 4,997,969 describe dithiocarbamate compounds and methods of making them.

Examples of suitable olefins that may be sulphurised to form an the sulphurised olefin include propylene, butylene, isobutylene, pentene, hexane, heptene, octane, nonene, decene, undecene, dodecene, undecyl, tridecene, tetradecene, pentadecene, hexadecene, heptadecene, octadecene, octadecenene, nonodecene, eicosene or mixtures thereof. In one embodiment, hexadecene, heptadecene, octadecene, octadecenene, nonodecene, eicosene or mixtures thereof and their dimers, trimers and tetramers are especially useful olefins. Alternatively, the olefin may be a Diels-Alder adduct of a diene such as 1,3-butadiene and an unsaturated ester such as butyl(meth)acrylate.

Another class of sulphurised olefin includes fatty acids and their esters. The fatty acids are often obtained from vegetable oil or animal oil and typically contain 4 to 22 carbon atoms. Examples of suitable fatty acids and their esters include triglycerides, oleic acid, linoleic acid, palmitoleic acid or mixtures thereof. Often, the fatty acids are obtained from lard oil, tall oil, peanut oil, soybean oil, cottonseed oil, sunflower seed oil or mixtures thereof. In one embodiment fatty acids and/or ester are mixed with olefins.

In another embodiment, the ashless antiwear agent may be a monoester of a polyol and an aliphatic carboxylic acid, often an acid containing 12 to 24 carbon atoms. Often the monoester of a polyol and an aliphatic carboxylic acid is in the form of a mixture with a sunflower oil or the like, which may be present in the friction modifier mixture from 5 to 95, in several embodiments from 10 to 90, or 20 to 85, or 20 to 80 weight percent of said mixture. The aliphatic carboxylic acids (especially a monocarboxylic acid) which form the esters are those acids typically containing 12 to 24 or 14 to 20 carbon atoms. Examples of carboxylic acids include dodecanoic acid, stearic acid, lauric acid, behenic acid, and oleic acid.

Polyols include diols, triols, and alcohols with higher numbers of alcoholic OH groups. Polyhydric alcohols include ethylene glycols, including di-, tri- and tetraethylene glycols; propylene glycols, including di-, tri- and tetrapropylene glycols; glycerol; butane diol; hexane diol; sorbitol; arabitol; mannitol; sucrose; fructose; glucose; cyclohexane diol; erythritol; and pentaerythritols, including di- and tripeutaerythritol. Often the polyol is diethylene glycol, triethylene glycol, glycerol, sorbitol, pentaerythritol or dipentaerythritol.

The commercially available monoester known as “glycerol monooleate” is believed to include 60±5 percent by weight of the chemical species glycerol monooleate, along with 35±5 percent glycerol dioleate, and less than 5 percent trioleate and oleic acid.

Other performance additives such as corrosion inhibitors including octylamine octanoate, condensation products of dodecenyl succinic acid or anhydride and a fatty acid such as oleic acid with a polyamine; metal deactivators including derivatives of benzotriazoles, 1,2,4-triazoles, benzimidazoles, 2-alkyldithiobenzimidazoles or 2-alkyldithiobenzothiazoles; foam inhibitors including copolymers of ethyl acrylate and 2-ethylhexylacrylate and optionally vinyl acetate; demulsifiers including trialkyl phosphates, polyethylene glycols, polyethylene oxides, polypropylene oxides and (ethylene oxide-propylene oxide) polymers; pour point depressants including esters of maleic anhydride-styrene, polymethacrylates, polyacrylates or polyacrylamides; and friction modifiers including fatty acid derivatives such as amines, esters, epoxides, fatty imidazolines, condensation products of carboxylic acids and polyalkylene-polyamines and amine salts of alkylphosphoric acids may also be used in the lubricant or fuel composition.

The following examples provide an illustration of the invention. These examples are non exhaustive and are not intended to limit the scope of the invention.

### EXAMPLES

### Preparative Examples of Dispersions

A series of dispersions (Reference Examples 1 to 3; and Example 1) containing (i) at least one component, (ii) a powder, (iii) an organic medium and (iv) a surfactant, are prepared from a slurry weighing about 15 kg using a lab scale Dyno-Mill ECM Multi-Lab horizontal bead mill commercially available from W.A.B. A.G., Basel, using 0.3 mm Ø zirconia/yttria beads and a residence time of about 10 minutes at a tip speed of about 8 ms-¹. Further the milling time, and other processing conditions are substantially the same for all dispersions prepared. Where appropriate, the mean particle size of the dispersion particles is determined after cooling by Coulter® LS230 Particle Size Analyser. The dispersions prepared are pourable.

### Reference Example 1

### Magnesium Oxide Dispersion

A dispersion is prepared by milling about 50 wt % Magnesium oxide, Magchem 40 ex Martin Marietta, in the presence of about 40 wt % 100 N base oil and about 10 wt % of an alkyl benzene sulphonic acid surfactant. The dispersion particles have an average mean particle size of greater than about 0.46 microns

### Reference Example 2

### Iron Oxide Dispersion

A dispersion is prepared by milling about 70 wt % of iron oxide (Fe2O3) commercially available from Bayer as Bayferrox® 160, about 18 wt % of 100 N base oil and about 12 wt % of an alkyl benzene sulphonic acid surfactant. The dispersion particles have an average mean particle size of greater than about 0.46 microns

### Reference Example 3

### Cerium Oxide Dispersion

A dispersion is prepared by milling about 50 wt % of cerium oxide (CeO), about 40 wt % of 100 N base oil and about 10 wt % of a surfactant (polyolefin amino ester esterified with 2-(dimethylamino)ethanol). The dispersion particles have an average mean particle size of greater than about 0.46 microns.

### Reference Example 4

### Three Metal Dispersion

A three metal dispersion is prepared by blending portions of the products formed in Preparative Examples 1 to 3. The final product has a metal weight ratio of magnesium:cerium:iron of about 150:10:5. The product forms a stable dispersion that shows no significant stratification after 12 weeks. The dispersion has a dispersion average mean particle size of greater than about 0.34 to about 0.40 microns.

### Example 1

### Three Metal Dispersion

A three metal dispersion is prepared by blending in a powder form magnesium oxide, calcium hydroxide and iron oxide (Fe2O3). The resultant three metal powder is then added to about 10 wt % of a succinimide surfactant, and about 39.6 wt % of SN 100 base oil and about 0.4 wt % of a demulsifier. The final dispersion contains 37.5 wt % magnesium oxide, about 10.5 wt % calcium hydroxide and about 2 wt % iron oxide. The resultant dispersion is pourable and with a mean particle size of about 0.14 to about 0.2 microns.

A comparison of the mean particle size obtained for the invention (Example 1 of about 0.14 to about 0.2 microns) compared with the Reference Example 4 (mean particle size of greater than about 0.34 to about 0.40 microns), demonstrates that a dispersion prepared by the process of the invention has a lower mean particle size than Reference Example 4. Therefore the invention provides a process for preparing a dispersion with a lower mean particle size, or a reduced agitation time, or combinations thereof.

While the invention has been explained in relation to its preferred embodiments, it is to be understood that various modifications thereof will become apparent to those skilled in the art upon reading the specification. Therefore, it is to be understood that the invention disclosed herein is intended to cover such modifications as fall within the scope of the appended claims.

What is claimed is:

1. A process for preparing a dispersion of particles, comprising:
reducing in particle size at least one component by agitating the component in the presence of (i) a plurality of beads; (ii) a powder; (iii) a surfactant; and (iv) a liquid medium to form a dispersion, wherein the particles are uniformly dispersed in the liquid medium.

2. The process of claim 1, wherein agitating comprises wet or dry processes.

3. The process of claim 1, wherein the powder and the component are both inorganic.

4. The process of claim 1, wherein the powder is inorganic and the component is organic.

5. The process of claim 1, wherein the powder and component are both a metal base.

6. The process of claim 5 wherein the metal base comprises at least one of magnesium hydroxide, calcium hydroxide, calcium carbonate, magnesium carbonate, calcium oxide, magnesium oxide, cerium oxide, iron oxide, sodium carbonate, sodium bicarbonate, potassium carbonate, potassium bicarbonate, potassium hydroxide, sodium hydroxide, anhydrous lithium hydroxide, lithium hydroxide monohydrate, lithium carbonate, lithium oxide, copper acetate, or mixtures thereof.

7. The process of claim 1, wherein the component comprises at least one of lubricant or fuel technology additives, pharmaceutical active agents, agrochemical active agents, personal care active agents, or mixtures thereof.

8. The method of claim 1, wherein the surfactant has a hydrophilic lipophilic balance (HLB) ranging from about 1 to about 40.

9. The method of claim 1, wherein the surfactant comprises at least one of hydrocarbyl substituted aryl sulphonic acids, a polyolefin-substituted acylating agent, or salixarenes.

10. A composition comprising: (a) a dispersion of particles obtained/obtainable from the process of claim 1; and (b) at least one member of the group consisting of an oil of lubricating viscosity, a grease thickener a liquid fuel, other performance additives, and mixtures thereof.
